SEQUENCE LISTING (5 sequences)

SEQ ID NO: 1 - protein, 332 aa, Haemophilus Influenzae
  ref <301>: AUTHORS: Harrison,A., Dyer,D.W., Gillaspy,A., Ray,W.C., Mungur,R.,
  ref <302>: TITLE: Genomic Sequence of an Otitis Media Isolate of Nontypeable
  ref <303>: JOURNAL: J. Bacteriol.
  ref <304>: VOLUME: 187
  ref <305>: ISSUE: 13
  ref <306>: PAGES: 4627-36
  ref <307>: DATE: 2005-07-01
  ref <308>: DATABASE ACCESSION NUMBER: YP_247820
  ref <309>: DATABASE ENTRY DATE: 2006-01-18
  ref <313>: RELEVANT RESIDUES IN SEQ ID NO: (1)..(332)
  ref <308>: DATABASE ACCESSION NUMBER: YP_247820
  ref <309>: DATABASE ENTRY DATE: 2005-06-27
  ref <313>: RELEVANT RESIDUES IN SEQ ID NO: (1)..(332)
  ref <308>: DATABASE ACCESSION NUMBER: NCBI/YP_247820
  ref <309>: DATABASE ENTRY DATE: 2005-06-27
  ref <313>: RELEVANT RESIDUES IN SEQ ID NO: (1)..(332)
MQFKHFKLATLAAALAFSANSFADITVYNGQHKEAATAVAKAFEQETGIKVTLNSGKSEQLAGQLKEEGDKTPADVFYTEQTATFADLSEAGLLAPISEQTIQQTAQKGVPLAPKKDWIALSGRSRVVVYDHTKLSEKDMEKSVLDYATPKWKGKIGYVSTSGAFLEQVVALSKMKGDKVALNWLKGLKENGKLYAKNSVALQAVENGEVPAALINNYYWYNLAKEKGVENLKSRLYFVRHQDPGALVSYSGAAVLKASKNQAEAQKFVDFLASKKGQEALVAARAEYPLRADVVSPFNLEPYEKLEAPVVSATTAQDKEHAIKLIEEAGLK

SEQ ID NO: 2 - protein, 330 aa, Neisseria meningitidis
  ref <301>: AUTHORS: Berish,S.A., Kapczynski,D.R. and Morse,S.A
  ref <302>: TITLE: Nucleotide sequence of the Fbp gene from Neisseria meningitidis
  ref <303>: JOURNAL: Nucleic Acids Res.
  ref <304>: VOLUME: 18
  ref <305>: ISSUE: 15
  ref <306>: PAGES: 4596
  ref <307>: DATE: 1990-06-01
  ref <308>: DATABASE ACCESSION NUMBER: NCBI / CAA 37561
  ref <309>: DATABASE ENTRY DATE: 1990-06-01
  ref <313>: RELEVANT RESIDUES IN SEQ ID NO: (1)..(330)
  ref <308>: DATABASE ACCESSION NUMBER: CAA37561
  ref <309>: DATABASE ENTRY DATE: 1990-06-01
  ref <313>: RELEVANT RESIDUES IN SEQ ID NO: (1)..(330)
  ref <308>: DATABASE ACCESSION NUMBER: NCBI / CAA37561
  ref <309>: DATABASE ENTRY DATE: 1990-06-01
  ref <313>: RELEVANT RESIDUES IN SEQ ID NO: (1)..(330)
MKTSIRYALLAAALTAATPALADITVYNGQHKEAAQAVADAFTRATGIKVKLNSAKGDQLAGQIKEEGSRSPADVFYSEQIPALATLSAANLLEPLPASTINETRGKGVPVAAKKDWVALSGRSRVVVYDTRKLSEKDLEKSVLNYATPKWKNRIGYAPTSGAFLEQVVAIVKLKGEAAALKWLKALKEYGKPYAKNSVALQAVENGEIDAALINNYYWHAFAREKGVQNVHTRLNFVRHRDPGALVTYSGAVLKSSQNKDEAKKFVAFLAGKEGQRALTAVRAEYPLNPHVVSTFNLEPIAKLEAPQVSATTVSEKEHATRLLEQAGMK

SEQ ID NO: 3 - protein, 1435 aa, Homo sapiens
  ref <301>: AUTHORS: Matsuzaka,Y., Okamoto,K., Mabuchi,T., Iizuka,M., Ozawa, A., Oka,A.,
  ref <302>: TITLE: Identification, expression analysis and polymorphism of a novel
  ref <303>: JOURNAL: Gene
  ref <304>: VOLUME: 343
  ref <305>: ISSUE: 2
  ref <306>: PAGES: 291-304
  ref <307>: DATE: 2004-12-22
  ref <308>: DATABASE ACCESSION NUMBER: Pubmed / 15588584
  ref <309>: DATABASE ENTRY DATE: 2005-05-22
  ref <313>: RELEVANT RESIDUES IN SEQ ID NO: (1)..(1435)
  ref <301>: AUTHORS: Matsuzaka,Y., Okamoto,K., Mabuchi,T., Iizuka,M., Ozawa, A., Oka,A.,
  ref <302>: TITLE: Identification, expression analysis and polymorphism of a novel
  ref <303>: JOURNAL: Genes and Development
  ref <304>: VOLUME: 343
  ref <305>: ISSUE: 2
  ref <306>: PAGES: 291-304
  ref <307>: DATE: 2004-12-22
  ref <308>: DATABASE ACCESSION NUMBER: Pubmed / 15588584
  ref <309>: DATABASE ENTRY DATE: 2005-05-22
  ref <313>: RELEVANT RESIDUES IN SEQ ID NO: (1)..(1435)
  ref <301>: AUTHORS: Matsuzaka,Y., Okamoto,K., Mabuchi,T., Iizuka,M., Ozawa, A., Oka,A.,
  ref <302>: TITLE: Identification, expression analysis and polymorphism of a novel
  ref <303>: JOURNAL: Genes and Development
  ref <304>: VOLUME: 343
  ref <305>: ISSUE: 2
  ref <306>: PAGES: 291-304
  ref <307>: DATE: 2004-12-22
  ref <308>: DATABASE ACCESSION NUMBER: Pubmed / 15588584
  ref <309>: DATABASE ENTRY DATE: 2005-05-22
  ref <313>: RELEVANT RESIDUES IN SEQ ID NO: (1)..(1435)
  ref <308>: DATABASE ACCESSION NUMBER: NCBI /  NP_001013860
  ref <309>: DATABASE ENTRY DATE: 2005-05-22
  ref <313>: RELEVANT RESIDUES IN SEQ ID NO: (1)..(1435)
MAQTPDGISCELRGEITRFLWPKEVELLLKTWLPGEGAVQNHVLALLRWRAYLLHTTCLPLRVDCTFSYLEVQAMALQETPPQVTFELESLRELVLEFPGVAALEQLAQHVAAAIKKVFPRSTLGKLFRRPTPASMLARLERSSPSESTDPCSPCGGFLETYEALCDYNGFPFREEIQWDVDTIYHRQGCRHFSLGDFSHLGSRDLALSVAALSYNLWFRCLSCVDMKLSLEVSEQILHMMSQSSHLEELVLETCSLRGDFVRRLAQALAGHSSSGLRELSLAGNLLDDRGMTALSRHLERCPGALRRLSLAQTGLTPRGMRALGRALATNAAFDSTLTHLDLSGNPGALGASEDSGGLYSFLSRPNVLSFLNLAGTDTALDTVRGCSVGGWMTGRADWRAGRGGLGPPAGVANSLPPQLFAAVSRGCCTSLTHLDASRNVFSRTKSRAAPAALQLFLSRARTLRHLGLAGCKLPPDALRALLDGLALNTHLRDLHLDLSACELRSAGAQVIQDLVCDAGAVSSLDLADNGFGSDMVTLVLAIGRSRSLRHVALGRNFNVRCKETLDDVLHRIVQLMQDDDCPLQSLSVAESRLKLGASVLLRALATNPNLTALDISGNAMGDAGAKLLAKALRVNSRLRSVVWDRNHTSALGLLDVAQALEQNHSLKAMPLPLNDVAQAQRSRPELTARAVHQIQACLLRNNRADPASSDHTTRLQPLGLVSDPSEQEVNELCQSVQEHVELLGCGAGPQGEAAVRQAEDAIQNANFSLSILPILYEAGSSPSHHWQLGQKLEGLLRQVGEVCRQDIQDFTQATLDTARSLCPQMLQGSSWREQLEGVLAGSRGLPELLPEQLLQDAFTRLRDMRLSITGTLAESIVAQALAGLSAARDQLVESLAQQATVTMPPALPAPDGGEPSLLEPGELEGLFFPEEKEEEKEKDDSPPQKWPELSHGLHLVPFIHSAAEEAEPEPELAAPGEDAEPQAGPSARGSPSPAAPGPPAGPLPRMDLPLAGQPLRHPTRARPRPRRQHHHRPPPGGPQVPPALPQEGNGLSARVDEGVEEFFSKRLIQQDRLWAPEEDPATEGGATPVPRTLRKKLGTLFAFKKPRSTRGPRTDLETSPGAAPRTRKTTFGDLLRPPTRPSRGEELGGAEGDTSSPDPAGRSRPRYTRDSKAYSMILLPAEEEATLGARPDKRRPLERGETELAPSFEQRVQVMLQRIGVSRGSGGAEGKRKQSKDGEIKKAGSDGDIMDSSTEAPPISIKSRTHSVSADPSCRPGPGSQGPESATWKTLGQQLNAELRSRGWGQQDGPGPPSPGQSPSPCRTSPSPDSLGLPEDPCLGPRNEDGQLRPRPLSAGRRAVSVHEDQLQAPAERPLRLQRSPVLKRRPKLEAPPSPSLGSGLGTEPLPPQPTEPSSPERSPPSPATDQRGGGPNP

SEQ ID NO: 4 - protein, 11 aa, Artificial Sequence
  Peptide capable of binding integrins and comprising the RGD sequence
  DISULFID (2)..(8): disulfide bonds (2, 8).
  DISULFID (4)..(10): disulfide bond (4, 10).
  ref <301>: AUTHORS: Ruoslahti,E.
  ref <302>: TITLE: RGD and other recognition sequences for integrins
  ref <303>: JOURNAL: Annu Rev Cell Dev Biol
  ref <304>: VOLUME: 12
  ref <306>: PAGES: 697-715
  ref <307>: DATE: 1996
  ref <308>: DATABASE ACCESSION NUMBER: NCBI / 1FUV_A
  ref <309>: DATABASE ENTRY DATE: 2000-09-15
  ref <313>: RELEVANT RESIDUES IN SEQ ID NO: (1)..(11)
  ref <301>: AUTHORS: Assa-Munt,N., Jia,X., Laakkonen,P. and Ruoslahti,E.
  ref <302>: TITLE: Solution structures and integrin binding activities of an RGD
  ref <303>: JOURNAL: Biochemistry
  ref <304>: VOLUME: 40
  ref <305>: ISSUE: 6
  ref <306>: PAGES: 2373-2378
  ref <307>: DATE: 2001
  ref <308>: DATABASE ACCESSION NUMBER: NCBI / 1FUV_A
  ref <309>: DATABASE ENTRY DATE: 2000-09-15
  ref <313>: RELEVANT RESIDUES IN SEQ ID NO: (1)..(11)
  ref <308>: DATABASE ACCESSION NUMBER: NCBI / 1FUV_A
  ref <309>: DATABASE ENTRY DATE: 2000-09-15
  ref <313>: RELEVANT RESIDUES IN SEQ ID NO: (1)..(11)
ACDCRGDCFCG

SEQ ID NO: 5 - protein, 11 aa, Artificial Sequence
  Peptide capable of binding integrins and comprising the RGD sequence
  DISULFID (2)..(10): disulfide bond (2, 10).
  DISULFID (4)..(8): disulfide bond (4, 8).
  ref <301>: AUTHORS: Ruoslahti,E.
  ref <302>: TITLE: RGD and other recognition sequences for integrins
  ref <303>: JOURNAL: Annu Rev Cell Dev Biol
  ref <304>: VOLUME: 12
  ref <306>: PAGES: 697-715
  ref <307>: DATE: 1996-11
  ref <313>: RELEVANT RESIDUES IN SEQ ID NO: (1)..(11)
  ref <301>: AUTHORS: Assa-Munt,N., Jia,X., Laakkonen,P. and Ruoslahti,E.
  ref <302>: TITLE: Solution structures and integrin binding activities of an RGD
  ref <303>: JOURNAL: Biochemistry
  ref <304>: VOLUME: 40
  ref <305>: ISSUE: 8
  ref <306>: PAGES: 2373-2378
  ref <307>: DATE: 2001-02-27
  ref <313>: RELEVANT RESIDUES IN SEQ ID NO: (1)..(11)
  ref <308>: DATABASE ACCESSION NUMBER: NCBI / 1FUL_A
  ref <309>: DATABASE ENTRY DATE: 2000-09-15
  ref <313>: RELEVANT RESIDUES IN SEQ ID NO: (1)..(11)
ACDCRGDCFCG

(19) United States
(12) Patent Application Publication
(10) Pub. No.: US 2009/0252688 A1
(43) Pub. Date: Oct. 8, 2009

(54) Magnetic resonance contrast medium containing an iron-binding protein

(75) Inventor: Arne Hengerer, Erlangen (DE)

Correspondence Address: HARNESS, DICKEY & PIERCE, P.L.C., P.O.BOX 8910, RESTON, VA, 20195, US

(73) Assignee: SIEMENS AKTIENGESELLSCHAFT

(21) Appl. No.: 12/010,135

(22) Filed: Jan. 22, 2008

(30) Foreign Application Priority Data
Jan. 23, 2007 (DE) 10 2007 004 283.5

(51) Int. Cl. A61K 51/10; C07K 14/00; C07H 21/04

(52) U.S. Cl. 424/9.34; 530/350; 536/23.1

(57) ABSTRACT

An inventive principle of at least one embodiment is based on linking an iron-binding functionality in the form of a bacterial iron-binding protein to a binding element which specifically recognizes a biological structure, in order to increase a local detectable increase in the concentration of the contrast medium. In at least one embodiment of the invention, a magnetic resonance contrast medium is provided which is capable of binding by way of a binding element to a biological structure in the body of a mammal, the binding element including an isolated polypeptide. The polypeptide includes a first amino acid sequence of a bacterial iron-binding protein or a derivative thereof, wherein the bacterial iron-binding protein or said derivative thereof has an iron-binding activity. In at least one embodiment, the binding element can bind a protein. In at least one embodiment, the binding element can have a ligand for a cellular membrane protein, a ligand for a cellular glycoprotein, an antibody or an antigen-binding fragment of an antibody and/or can bind a tumor antigen.

### PRIORITY STATEMENT

The present application hereby claims priority under 35 U.S.C. §119 on German patent application number DE 10 2007 004 283.5 filed Jan. 23, 2007, the entire contents of which is hereby incorporated herein by reference.

### FIELD

Embodiments of the invention generally relate to a magnetic resonance contrast medium which has an iron-binding protein or a derivative thereof having iron-binding activity. Embodiments of the invention further generally relate to a corresponding isolated polypeptide, a corresponding isolated nucleic acid and/or a pharmaceutical composition which has the magnetic resonance contrast medium of an embodiment of the invention.

### BACKGROUND

Contrast media containing paramagnetic metals, for example iron, are known to be used in imaging processes using magnetic resonance (MR) or nuclear spin methods. In order to increase the efficacy of contrast media, specific contrast media are known to be used, which enables specific structures in the body to be recognized, for example by binding to a biological structure. This takes place via a local modification of the magnetic field, for example by the contrast medium having a paramagnetic or superparamagnetic signaling molecule. Specific contrast media are known to be used which firstly can bind to a biological structure (e.g. via antigen-antibody binding) and secondly have a paramagnetic metal. Kuriu et al., for example, disclose a contrast medium which has a monoclonal antibody conjugated to a paramagnetic metal (Kuriu Y, Otsuji E, Kin S, Nakase Y, Fukuda K I, Okamoto K, Hagiwara A, Hamagishi H; Monoclonal antibody conjugated to gadolinium as a contrast agent for magnetic resonance imaging of human rectal carcinoma. J Surg Oncol. 2006 Jul. 17; 94(2):144-148).

Paramagnetic or superparamagnetic iron oxide nanoparticles which can be functionalized have also proved to be particularly suitable MR contrast media (H, Lee E, Kim do K, Jang N K, Jeong Y Y, Jon S., Antibiofouling polymer-coated superparamagnetic iron oxide nanoparticles as potential magnetic resonance contrast agents for in vivo cancer imaging. J Am Chem Soc. 2006 Jun 7:128(22):7383-9).

### SUMMARY

In at least one embodiment of the present invention, a magnetic resonance contrast medium is provided which is suitable for binding biological structures specifically and which binds iron in a particularly efficient manner.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described. Like numbers refer to like elements throughout. As used herein, the terms “and/or” and “at least one of” include any and all combinations of one or more of the associated listed items.

In an embodiment of the invention, a magnetic resonance contrast medium is provided which can bind, by way of a binding element, to a biological structure in the body of a mammal, wherein the binding element has an isolated polypeptide, wherein the polypeptide comprises a first amino acid sequence of a bacterial iron-binding protein or a derivative thereof, wherein said bacterial iron-binding protein or said derivative thereof has an iron-binding activity.

It is noted here that the term “iron-binding” is not limited to binding of elemental iron but comprises binding of the element iron in any form, as element, as iron, as oxide, in the form of particles or nanoparticles, etc.

A polypeptide has a series of amino acids having a defined sequence. An isolated polypeptide means a polypeptide which is in a form that is essentially free of other polypeptides.

A binding element which binds to a biological structure in the body of a mammal is any element that binds to a biological structure with sufficient specificity and affinity so as to enable the contrast agent to be accumulated in a detectable manner at the site at which the biological structure is located. A biological structure refers to any endogenous structure accessible to binding by the binding element. A non-conclusive list of examples of the binding element comprises antibodies, ligands for receptors, ligands for cellular membrane proteins, ligands for glycoproteins, wherein said ligands may be both of natural origin (for example the natural ligand or a modified form of the natural ligand of a receptor) and of artificial origin; antibody fragments, single-chain antibodies, etc. Examples of the biological structure comprise (non-conclusively) proteins, in particular membrane proteins, glycoproteins, carbohydrates, nucleic acids, lipoproteins, tumor antigens, etc.

A bacterial iron-binding protein refers to a protein which binds iron with high specificity (e.g. binding constant of >10¹⁵ mol⁻¹, preferably >10¹⁸ mol⁻¹) and which is of bacterial origin. A derivative of a bacterial iron-binding protein refers to a polypeptide derived from the natural protein, which has sequence homology to said natural protein and which itself has an iron-binding activity with a high binding constant (e.g. binding constant of >10¹⁵ mol⁻¹, preferably >10¹⁸ mol⁻¹).

Iron is an important trace element for living organisms, this firstly being a consequence of the abundance of this element in the environment and secondly resulting from its chemical properties, since iron possesses two stable oxidation states (+II/+III) which can be converted into one another and which are well suited to participate in redox processes, for example within the respiratory chain. Iron may occur in proteins in different structures, for example as heme group, as iron-sulfur cluster, as iron-nickel, as diiron or as mononuclear iron. As a cofactor of many enzymes, it is involved in important metabolic processes. It is, however, difficult to access for cells, due to the low solubility products of Fe(II) and Fe(III). Moreover, Fe(III) hydrolyzes in an aqueous environment and forms polymeric hydroxides which can precipitate under physiological conditions. Since iron moreover catalyzes the formation of free radicals, it has additionally highly toxic effects. For this reason, availability of iron in the body is highly regulated, with iron being bound by endogenous iron-binding proteins, for example transferring, ferritins, and being able to be transported to the cell interior.

Iron is a valuable resource for bacteria which colonize the body of a mammal. For this reason, bacteria have evolved a multiplicity of iron-binding proteins which for their part serve to bind iron and make it available for bacteria. These bacterial iron-binding proteins are distinguished by extremely high iron constants, for example >10¹⁵ mol⁻¹ or >10¹⁸ mol⁻¹ (Briat J.-F. (1992). Iron assimilation and storage in prokaryotes. J. Gen. Microbiol. 138: 2475-2483, Guerinot, M. L. (1994) Microbial iron transport. Annu Rev Microbiol (48):743-772). Consequently, bacterial iron-binding proteins are extremely efficient iron chelators.

The inventive principle of at least one embodiment is based on linking an iron-binding functionality in the form of a bacterial iron-binding protein to a binding element which specifically recognizes a biological structure, in order to increase a local detectable increase in the concentration of the contrast medium. Preferably, the binding element can bind a protein. Particularly preferably, the binding element can have a ligand for a cellular membrane protein, a ligand for a cellular glycoprotein, an antibody or an antigen-binding fragment of an antibody and/or can bind a tumor antigen.

According to an example aspect of an embodiment of the invention, the binding element comprises a second amino acid sequence.

The binding element is linked to the iron-binding functionality, for example, by a covalent bond or in the form of a stable complex.

According to an example aspect of an embodiment of the invention, the second amino acid sequence encompassed by the binding element preferably forms part of the polypeptide which likewise comprises the first amino acid sequence which has a bacterial iron-binding protein or a derivative thereof. Thus, according to this example aspect, the polypeptide comprises both the binding element and the iron-binding functionality.

The polypeptide, in an embodiment, preferably has a spacer or linker amino acid sequence which is designed to be located between the first and second amino acid sequences. The spacer amino acid sequence may be, for example, from 10 to 20 amino acids in length, with common amino acid sequences suitable for use as spacer amino acid sequence being known to the skilled worker, for example polyglycine sequences and the like.

Preference, in an embodiment, is given to the bacterial iron-binding protein being a siderophore. Siderophores are high affinity extracellular iron(III) chelators.

Preference, in an embodiment, is given to the bacterial iron-binding protein being an Fe(III)-binding protein (Fbp) or a major ferric binding protein (MIRP) of the Haemophilus, Pasteurellales, Pasteurellaceae or Neisseria families. More preference, in an embodiment, is given to the bacterial iron-binding protein being an Fe(III)-binding protein (Fbp) of the species H. influenzae, N. gonorrohoeae, N. meningitidis, N. cinerea, N. lactamica, N. subflava, N. kochii or N. polysaccharea.

According to an example aspect of an embodiment of the invention, the first amino acid sequence is chosen from the group consisting of:
a) the amino acid sequence according to SEQ ID NO:1 or SEQ ID NO:2;
b) an amino acid sequence having at least 15, preferably 30, contiguous amino acids of the amino acid sequence according to SEQ ID NO:1 or SEQ ID NO:2;
c) an amino acid sequence of a derivative of a polypeptide having the amino acid sequence according to SEQ ID NO:1 or SEQ ID NO:2, wherein said derivative is encoded by a nucleic acid molecule which hybridizes under stringent conditions to a nucleic acid molecule which encodes a polypeptide having the amino acid sequence according to SEQ ID NO:1 or SEQ ID NO:2;
d) an amino acid sequence which is at least 60% homologous to the amino acid sequence according to SEQ ID NO:1 or SEQ ID NO:2; and
e) an amino acid sequence of a derivative of a polypeptide having the amino acid sequence according to SEQ ID NO:1 or SEQ ID NO:2, wherein said derivative is encoded by a nucleic acid molecule which is at least 60% homologous to a nucleic acid molecule which encodes said polypeptide having the amino acid sequence according to SEQ ID NO:1 or SEQ ID NO:2.

In the context of an embodiment of the present invention, stringent hybridization conditions refers to conditions which make possible hybridization of allelic variants but do not make possible hybridization with other nonrelated genes. The usual conditions known to the skilled worker, as described in Sambrook et al. (Molecular Cloning. A laboratory manual, Cold Spring Harbour Laboratory Press, 2nd edition, 1989) are used here. Stringent hybridization conditions are, for example, 6×sodium chloride/sodium citrate (SSC) at about 45° C., followed by a washing step with 2×SSC at 50° C.

In the context of an embodiment of the present invention, the expression “homologous” means a defined homology of at least 60%, preferably 75%, more preferably 90%, at the DNA or amino acid level, which can be determined by known methods, for example by computer-assisted sequence comparisons. This involves comparing two sequences to be investigated with respect to their homology in a computer-assisted manner (for example, S. F. Altschul et al. (1999), basic local alignment search tool, J. Mol. Biol. 215 or by the “Global Alignment Program” (GAP) of the Genetics Computer Group (GCG)), so as to produce the greatest possible match (“alignment”), with the number of matching nucleotides or amino acids then being expressed as percentage of the total number of nucleotides and amino acids, respectively, in the sequence.

Preferably, in an embodiment, the second amino acid sequence which is encompassed by the binding element has the amino acid sequence ARG-GLY-ASP (RGD).

Proteins comprising the ARG-GLY-ASP (RGD) binding site can bind integrins which are expressed, for example, on endothelial cells of blood vessels. The RGD sequence is the binding site for a large number of adhesion proteins of extracellular matrix, blood and cell surface, and the integrin-binding activity of adhesion proteins can be reproduced by short synthetic peptides which comprise the RGD sequence. Such peptides assist adhesion to cells, and it is also possible to choose RGD sequences which specifically bind to particular integrins. See also Ruoslahti, E., Annu. Rev. Cell. Dev. Biol., 1996; 12; 697-715.

Binding elements comprising the RGD sequence may bind the contrast medium specifically to cellular integrins. Two examples of peptides capable of binding integrins and comprising the RGD sequence are given by way of SEQ ID NO:4 and SEQ ID NO:5. According to one embodiment of the present invention, preference is given to the second amino acid sequence comprising the sequences according to SEQ ID NO:4 or SEQ ID NO:5.

Further peptides having the RGD binding motif, which are suitable as binding element, are described also in the published patent application US 20050070466A1, for example.

SEQ ID NO:3 describes by way of example an integrin-binding protein having the RGD motif (see also Matsuzaka Y, Okamoto K, Mabuchi T, Iizuka M, Ozawa A, Oka A, Tamiya G, Kulski J K, Inoko H., Identification, expression analysis and polymorphism of a novel RLTPR gene encoding an RGD motif, tropomodulin domain and proline/leucine-rich regions. Gene. 2004 Dec. 22;343(2):291-304).

According to a further aspect of an embodiment of the present invention, preference is given to the second amino acid sequence being chosen from the group consisting of:
a) the amino acid sequence according to SEQ ID NO:3;
b) an amino acid sequence which has at least 15, preferably 30, contiguous amino acids of the amino acid sequence according to SEQ ID NO:3;
c) an amino acid sequence of a derivative of a polypeptide having the amino acid sequence according to SEQ ID NO:3, wherein said derivative is encoded by a nucleic acid molecule which under stringent conditions hybridizes to a nucleic acid molecule which encodes said polypeptide having the amino acid sequence according to SEQ ID NO:3;
d) an amino acid sequence which is at least 60% homologous to the amino acid sequence according to SEQ ID NO:3; and
e) an amino acid sequence of a derivative of a polypeptide having the amino acid sequence according to SEQ ID NO:3, wherein said derivative is encoded by a nucleic acid molecule which is at least 60% homologous to a nucleic acid molecule which encodes said polypeptide having the amino acid sequence according to SEQ ID NO:3.

A derivative of the polypeptide refers to a polypeptide derived from the natural protein, which has sequence homology to said natural protein and which itself acts as binding element, i.e. binds to the particular biological structure.

An embodiment of the invention further relates to an isolated polypeptide having a first amino acid sequence and a second amino acid sequence according to the above-described variants. An isolated polypeptide means a polypeptide which is in a form that is essentially free of other polypeptides.

In addition, the polypeptide may have further elements or sequence regions, for example a linker sequence or markers for purifying the polypeptide.

The polypeptide of an embodiment of the invention has a first domain having a first amino acid sequence of a bacterial iron-binding protein or a derivative of said first amino acid sequence, which derivative has an iron-binding activity, said polypeptide having a second domain which has a second amino acid sequence and which acts as binding element for binding to a biological structure in the body of a mammal.

Moreover, an embodiment of the invention relates to an isolated nucleic acid molecule comprising a nucleic acid sequence which encodes the polypeptide of an embodiment of the invention. The nucleic acid sequence may be present in a suitable vector, for example an expression plasmid suitable for a particular expression system. An isolated nucleic acid molecule refers to a nucleic acid molecule which is in a form that is essentially free of other nucleic acids.

An embodiment of the invention further relates to a pharmaceutical composition which has the magnetic resonance contrast medium of the invention in a pharmaceutically usable form and a pharmaceutically usable carrier.

### EXAMPLE OF AN EMBODIMENT OF THE INVENTION

Construction of the polypeptide

The polypeptide of an embodiment of the invention is prepared by preparing a fusion protein which firstly has the integrin-binding peptide according to SEQ ID NO:4 or SEQ ID NO:5 and secondly the amino acid sequence according to SEQ ID NO:1. The amino acid sequences SEQ ID NO:4 and SEQ ID NO:5 are integrin-binding peptides which have the same amino acid sequence with differently linked disulfide bridges. The domain which has the integrin-binding peptide can be spaced from the domain which has the iron-binding protein by a linker or spacer of, for example, 10-20 amino acids in length.

A nucleic acid which encodes the fusion peptide having the integrin-binding domain, the spacer and the iron-binding domain may be cloned in a suitable expression vector according to conventional cloning techniques known to the skilled worker (cf. Sambrook et al., Molecular Cloning. A laboratory manual, Cold Spring Harbour Laboratory Press, 2nd edition, 1989). The polypeptide may be expressed in a suitable expression system (E. coli, Baculovirus, CHO cells or the like). The polypeptide is then purified by way of a suitable process. To facilitate purification, it is conceivable to equip the polypeptide with further functionalities, for example a marker by which the protein can be purified by means of affinity chromatography, such as, for example, a polyhistidine residue, the “His-Tag”, or other markers known to the skilled worker.

Preparation of the contrast medium

Preparing the contrast medium involves firstly preparing a dispersion of monocrystalline or monodisperse iron oxide particles (nanoparticles of from 1 to 100 nm in diameter). The dispersion is prepared in a one-step synthesis from iron(II) and iron(III) salts. An NH₃ solution or NaOH solution is added with stirring to an aqueous iron chloride solution (FeCl₂ and FeCl₃). Magnetite, Fe₃O₄, precipitates from the solution. Oleic acid is added with stirring and heating to the suspension, resulting in finely dispersed magnetite particles (cf. also Park et al. (2005), One-nanometer-scale-size-controlled synthesis of monodisperse magnetic iron oxide nanoparticles, Angewandte Chemie International Edition, 44, 19, 2872-2877).

After purification, the particles are coated in a second step with the fusion proteins which are added by titration in aqueous solution with stirring to the iron suspension. Finally, still unoccupied surfaces of the iron nanoparticles must be saturated, for example with dextran, PEG, starch or the like. After purification, the coated particles are taken up in aqueous, pH-buffered solution. The iron concentration of the contrast medium should be, for example, from 0.1 mmol to 1.0 mmol of Fe/ml.

Use of the contrast medium

The contrast medium is administered by way of a 1-2 ml bolus injection. During the accumulation phase of from a few minutes to up to 8 hours after injection, an MR study may be carried out, with the use of a dynamic MRT imaging process, for example by means of T2*-weighted or T1-weighted gradient echo sequences (GRE), being recommended.

It is emphasized here that the example embodiment shown is merely by way of illustration and by way of example. Multiple variations and modifications are conceivable within the scope of the invention, in particular with regard to the choice of the first and, optionally, of the second amino acid sequence, linkage of the iron-binding functionality and the binding element and also with regard to the particular dosage form of the contrast medium.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A magnetic resonance contrast medium capable of binding by way of a binding element to a biological structure in a body of a mammal, the binding element including an isolated polypeptide, the polypeptide comprising a first amino acid sequence of a bacterial iron-binding protein or a derivative thereof, and the bacterial iron-binding protein or said derivative thereof including an iron-binding activity.

2. The magnetic resonance contrast medium as claimed in claim 1, wherein the binding element binds to a protein.

3. The magnetic resonance contrast medium as claimed in claim 2, wherein the binding element includes a ligand for a cellular membrane protein.

4. The magnetic resonance contrast medium as claimed in claim 2, wherein the binding element includes a ligand for a cellular glycoprotein.

5. The magnetic resonance contrast medium as claimed in claim 1, wherein the binding element includes an antibody or an antigen-binding fragment of an antibody.

6. The magnetic resonance contrast medium as claimed in claim 1, wherein the binding element binds to a tumor antigen.

7. The magnetic resonance contrast medium as claimed in claim 1, wherein the binding element binding to the biological structure includes a second amino acid sequence.

8. The magnetic resonance contrast medium as claimed in claim 7, wherein the second amino acid sequence encompassed by the binding element forms part of the polypeptide.

9. The magnetic resonance contrast medium as claimed in claim 8, wherein the polypeptide includes a linker amino acid sequence, designed to be located between the first and second amino acid sequences.

10. The magnetic resonance contrast medium as claimed in claim 1, wherein the bacterial iron-binding protein is a siderophore.

11. The magnetic resonance contrast medium as claimed in claim 1, wherein the bacterial iron-binding protein is an Fe(III)-binding protein (Fbp) or a major ferric binding protein (MIRP) of the Haemophilus, Pasteurellales, Pasteurellaceae or Neisseria family.

12. The magnetic resonance contrast medium as claimed in claim 1, wherein the bacterial iron-binding protein is an Fe(III)-binding protein (Fbp) of the species H. influenzae, N. gonorrohoeae, N. meningitidis, N. cinerea, N. lactamica, N. subflava, N. kochii or N. polysaccharea.

13. The magnetic resonance contrast medium as claimed in claim 1, wherein the first amino acid sequence is selected from the group consisting of:
a) the amino acid sequence according to SEQ ID NO:1 or SEQ ID NO:2;
b) an amino acid sequence having at least 15 contiguous amino acids of the amino acid sequence according to SEQ ID NO:1 or SEQ ID NO:2;
c) an amino acid sequence of a derivative of a polypeptide having the amino acid sequence according to SEQ ID NO:1 or SEQ ID NO:2, wherein said derivative is encoded by a nucleic acid molecule which hybridizes under stringent conditions to a nucleic acid molecule which encodes said polypeptide having the amino acid sequence according to SEQ ID NO:1 or SEQ ID NO:2;
d) an amino acid sequence which is at least 60% homologous to the amino acid sequence according to SEQ ID NO:1 or SEQ ID NO:2; and
e) an amino acid sequence of a derivative of a polypeptide having the amino acid sequence according to SEQ ID NO:1 or SEQ ID NO:2, wherein said derivative is encoded by a nucleic acid molecule which is at least 60% homologous to a nucleic acid molecule which encodes said polypeptide having the amino acid sequence according to SEQ ID NO:1 or SEQ ID NO:2.

14. The magnetic resonance contrast medium as claimed in claim 7, wherein the second amino acid sequence has the amino acid sequence Arg-Gly-Asp (RGD).

15. The magnetic resonance contrast medium as claimed in claim 14, wherein the second amino acid sequence comprises the sequence according to SEQ ID NO:4 or SEQ ID NO:5.

16. The magnetic resonance contrast medium as claimed in claim 1, wherein the second amino acid sequence is chosen from the group consisting of:
a) the amino acid sequence according to SEQ ID NO:3;
b) an amino acid sequence which has at least 15 contiguous amino acids of the amino acid sequence according to SEQ ID NO:3;
c) an amino acid sequence of a derivative of a polypeptide having the amino acid sequence according to SEQ ID NO:3, wherein said derivative is encoded by a nucleic acid molecule which under stringent conditions hybridizes to a nucleic acid molecule which encodes said polypeptide having the amino acid sequence according to SEQ ID NO:3;
d) an amino acid sequence which is at least 60% homologous to the amino acid sequence according to SEQ ID NO:3; and
e) an amino acid sequence of a derivative of a polypeptide having the amino acid sequence according to SEQ ID NO:3, wherein said derivative is encoded by a nucleic acid molecule which is at least 60% homologous to a nucleic acid molecule which encodes said polypeptide having the amino acid sequence according to SEQ ID NO:3.

17. An isolated polypeptide, comprising:
a first domain having a first amino acid sequence of a bacterial iron-binding protein or a derivative thereof which has an iron-binding activity; and
a second domain including a second amino acid sequence, which acts as binding element for binding to a biological structure in the body of a mammal.

18. An isolated nucleic acid molecule which encodes a polypeptide as claimed in claim 17.

19. A pharmaceutical composition having a magnetic resonance contrast medium as claimed in claim 1 and a pharmaceutically usable carrier.

20. The magnetic resonance contrast medium as claimed in claim 3, wherein the binding element includes a ligand for a cellular glycoprotein.

21. The magnetic resonance contrast medium as claimed in claim 2, wherein the bacterial iron-binding protein is a siderophore.

22. The magnetic resonance contrast medium as claimed in claim 2, wherein the bacterial iron-binding protein is an Fe(III)-binding protein (Fbp) or a major ferric binding protein (MIRP) of the Haemophilus, Pasteurellales, Pasteurellaceae or Neisseria family.

23. The magnetic resonance contrast medium as claimed in claim 2, wherein the bacterial iron-binding protein is an Fe(III)-binding protein (Fbp) of the species H. influenzae, N. gonorrohoeae, N. meningitidis, N. cinerea, N. lactamica, N. subflava, N. kochii or N. polysaccharea.

24. The magnetic resonance contrast medium as claimed in claim 7, wherein the first amino acid sequence is selected from the group consisting of:
a) the amino acid sequence according to SEQ ID NO:1 or SEQ ID NO:2;
b) an amino acid sequence having at least 15 contiguous amino acids of the amino acid sequence according to SEQ ID NO:1 or SEQ ID NO:2;
c) an amino acid sequence of a derivative of a polypeptide having the amino acid sequence according to SEQ ID NO:1 or SEQ ID NO:2, wherein said derivative is encoded by a nucleic acid molecule which hybridizes under stringent conditions to a nucleic acid molecule which encodes said polypeptide having the amino acid sequence according to SEQ ID NO:1 or SEQ ID NO:2;
d) an amino acid sequence which is at least 60% homologous to the amino acid sequence according to SEQ ID NO:1 or SEQ ID NO:2; and
e) an amino acid sequence of a derivative of a polypeptide having the amino acid sequence according to SEQ ID NO:1 or SEQ ID NO:2, wherein said derivative is encoded by a nucleic acid molecule which is at least 60% homologous to a nucleic acid molecule which encodes said polypeptide having the amino acid sequence according to SEQ ID NO:1 or SEQ ID NO:2.

25. The magnetic resonance contrast medium as claimed in claim 24, wherein the second amino acid sequence has the amino acid sequence Arg-Gly-Asp (RGD).

26. The magnetic resonance contrast medium as claimed in claim 25, wherein the second amino acid sequence comprises the sequence according to SEQ ID NO:4 or SEQ ID NO:5.

27. A pharmaceutical composition having a magnetic resonance contrast medium as claimed in claim 13 and a pharmaceutically usable carrier.

28. A pharmaceutical composition having a magnetic resonance contrast medium as claimed in claim 16 and a pharmaceutically usable carrier.
